(19) United States
(12) Patent Application Publication
(10) Pub. No.: US 2021/0084893 A1
(43) Pub. Date: Mar. 25, 2021

(54) FROZEN CELL CLUSTER AND METHOD FOR PRODUCING FROZEN CELL CLUSTER

(71) Applicant: Hiroshima University, Hiroshima (JP)

(72) Inventors: Hidemi Kurihara, Hiroshima-shi, Hiroshima (JP); Mikihito Kajiya, Hiroshima-shi, Hiroshima (JP); Souta Motoike, Hiroshima-shi, Hiroshima (JP)

(21) Appl. No.: 16/095,907

(22) PCT Filed: Apr. 7, 2017
(86) PCT No.: PCT/JP2017/014545

§ 371 Date: Oct. 23, 2018

(30) Foreign Application Priority Data
Apr. 27, 2016 (JP) 2016-089188

(51) Int. Cl. A01N 1/02; C12N 5/0775; C12N 5/00; A61L 27/38

(52) U.S. Cl. CPC A01N 1/0284; C12N 5/0663; C12N 2513/00; A61L 27/3895; A61L 27/3834; C12N 5/0062

(57) ABSTRACT

A frozen cell cluster is a cell cluster in a frozen state in which mesenchymal stem cells are granularly formed. A method for producing the frozen cell cluster includes culturing a mesenchymal stem cell in a growth medium containing a factor that causes the mesenchymal stem cell to produce collagen, forming a granular cell cluster containing the collagen produced from the mesenchymal stem cell, and isolating the cell cluster from the growth medium and cryopreserving the cell cluster together with a cryopreservation agent.

### TECHNICAL FIELD

The present disclosure relates to a frozen cell cluster and a method for producing a frozen cell cluster.

### BACKGROUND ART

A mesenchymal stem cell (MSC) has pluripotency and self-proliferation potency, and does not require gene transfer or the like, and is reliably available from a patient, so that an MSC is a cell suitable for tissue regeneration therapy.

For example, for osteoclastic diseases such as bone fracture and periodontitis, current MSC transplantation therapy isolates an MSC from a patient, allows growth, obtains a sufficient number of cells, and then cryopreserves the cells. After that, immediately before transplant surgery in the patient, the cryopreserved MSC is thawed, then cultured in a cell processing center (CPC), and processed into the form of an MSC graft in which the MSC assumes the form of a cluster.

For example, the thawed MSC is mixed with an artificial scaffold material (such as hydroxyapatite, tricalcium phosphate hydroxyapatite, polylactic acid, chitosan, atelocollagen gel, or hyaluronic acid gel) and cultured to produce an MSC graft. Then, the MSC graft is transplanted into the defective tissue of the patient to achievement of the regeneration ability of the MSC.

In addition, recently, there are MSC grafts that require no artificial scaffold material, and such MSC grafts include, in addition to cell sheets and cell spheroids, a cell cluster (Non-Patent Literature 1).

### CITATION LIST

### Non-Patent Literature

Non-Patent Literature 1: Kittaka M., Kajiya M., Shiba H., Takewaki M., Takeshita K., Khung R., Fujita T., Iwata T., Nguyen T.Q., Ouhara K., Takeda K., Fujita T., Kurihara H; “Clumps of a mesenchymal stromal cell/extracellular matrix complex can be a novel tissue engineering therapy for bone regeneration”; International Society for Cellular Therapy; Cytotherapy, 2015, 17, 860-873.

### SUMMARY OF INVENTION

### Technical Problem

Conventional MSC grafts are produced after thawing the previously cryopreserved MSCs immediately before transplant surgery. Since an MSC graft is produced each time within a limited period immediately before transplant surgery, there is no guarantee that the number of cells included in the MSC graft and functions of the cells can be made uniform. Due to this, when a desired MSC graft cannot be produced, transplant surgery may be postponed.

In consideration of the above circumstances, an objective of the present disclosure is to provide a frozen cell cluster that can be cryopreserved in a cluster form and a method for producing the frozen cell cluster.

### Solution to Problem

A frozen cell cluster according to a first aspect of the present disclosure is a cell cluster in a frozen state, in which mesenchymal stem cells are granularly formed.

Additionally, the frozen cell cluster preferably has a granular shape with a diameter of 0.5 mm to 1.5 mm.

Additionally, the frozen cell cluster preferably has a granular shape with a diameter of 0.8 mm to 1.2 mm.

A method for producing a frozen cell cluster according to a second aspect of the present disclosure includes:
culturing a mesenchymal stem cell in a growth medium containing a factor that causes the mesenchymal stem cell to produce collagen;
forming a granular cell cluster containing the collagen produced from the mesenchymal stem cell; and
isolating the cell cluster from the growth medium and cryopreserving the cell cluster together with a cryopreservation agent.

A method for producing a frozen cell cluster according to a third aspect of the present disclosure includes:
culturing a mesenchymal stem cell in a growth medium containing a factor that causes the mesenchymal stem cell to produce collagen to form a cell sheet;
making a periphery of the cell sheet a free edge to form a granular cell cluster by a self-aggregating effect; and
isolating the cell cluster from the growth medium and cryopreserving the cell cluster together with a cryopreservation agent.

In addition, the periphery of the cell sheet may be made the free edge by culturing the mesenchymal stem cell in a culture vessel and allowing growth of the mesenchymal stem cell to reach confluence, and separating an edge of the cell sheet adhering to a peripheral wall of the culture vessel from the peripheral wall of the culture vessel.

The culture vessel may have a cylindrical shape with an area of 1.5 cm² to 2.5 cm², and the obtained cell cluster may have a granular shape with a diameter of 0.5 mm to 1.5 mm.

A method for producing a frozen cell cluster according to a fourth aspect of the present disclosure includes:
culturing a mesenchymal stem cell in a growth medium to cause aggregation;
supplementing the growth medium with a factor that causes the mesenchymal stem cell to produce collagen to form a granular cell cluster containing the collagen produced from the mesenchymal stem cell; and
isolating the cell cluster from the growth medium and cryopreserving the cell cluster together with a cryopreservation agent.

The cell cluster may be cryopreserved without pre-freezing.

### Advantageous Effects of Invention

In the frozen cell cluster according to the present disclosure, MSCs are frozen in the cluster form, so that production of an MSC graft within a limited period immediately before transplant surgery or the like is unnecessary. Such configuration can prevent a situation in which transplant surgery is postponed.

### BRIEF DESCRIPTION OF THE DRAWINGS

FIG. 1 is a graph illustrating measurement results of cell survival activity;

FIG. 2 includes images (A) to (D) illustrating phase-contrast microscopic images of C-MSCs, in which image (A) is an image of non-cryopreserved C-MSCs, image (B) is an image of C-MSCs cryopreserved in a DMSO cryopreservation solution, image (C) is an image of C-MSCs cryopreserved in BAMBANKER (trade name, manufactured by Wako Pure Chemical Industries, Ltd.), and image (D) is an image of C-MSCs cryopreserved in CELL BANKER (trade name, manufactured by Takara Bio Inc.);

FIG. 3 includes images (A) to (F) illustrating HE-stained images of a C-MSC, in which image (A) is an image before freezing, image (B) is a partially enlarged image of image (A), image (C) is an image 1 day after thawing, image (D) is a partially enlarged image of image (C), image (E) is an image 5 days after thawing, and image (F) is a partially enlarged image of image (E);

FIG. 4 includes images (A) to (F) illustrating HE-stained images of a cell sheet, in which image (A) is an image before freezing, image (B) is a partially enlarged image of image (A), image (C) is an image 1 day after thawing, image (D) is a partially enlarged image of image (C), image (E) is an image 5 days after thawing, and image (F) is a partially enlarged image of image (E);

FIG. 5 includes images (A) to (F) illustrating HE-stained images of a cell spheroid, in which image (A) is an image before freezing, image (B) is a partially enlarged image of image (A), image (C) is an image 1 day after thawing, image (D) is a partially enlarged image of image (C), image (E) is an image 5 days after thawing, and image (F) is a partially enlarged image of image (E);

FIG. 6 is a graph illustrating percentages of cell death in C-MSCs before freezing and in C-MSCs, a cell sheet, and a cell spheroid 5 days after freezing and thawing;

FIG. 7 includes images (A) to (H) illustrating states of C-MSCs before cryopreservation, in which the C-MSCs were not supplemented with collagenase in the states illustrated in images (A) to (D), the C-MSCs were supplemented with collagenase in the states illustrated in images (E) to (H), image (A) is an image of HE staining, image (B) is an image of TUNNEL/DAPI staining, image (C) is a partially enlarged image of image (B), image (D) is an image of immunostaining, image (E) is an image of HE staining, image (F) is an image of TUNNEL/DAPI staining, image (G) is a partially enlarged image of image (F), and image (H) is an image of immunostaining;

FIG. 8 includes images (A) to (F) illustrating states of C-MSCs 5 days after cryopreservation and thawing, in which the C-MSCs were not supplemented with collagenase in the states illustrated in images (A) to (C), the MSCs were supplemented with collagenase in the states illustrated in images (D) to (F), image (A) is an image of HE staining, image (B) is an image of TUNNEL/DAPI staining, image (C) is a partially enlarged image of image (B), image (D) is an image of HE staining, image (E) is an image of TUNNEL/DAPI staining, and image (F) is a partially enlarged image of image (E);

FIG. 9 is a graph illustrating percentages of cell death in C-MSCs 5 days after thawing; FIG. 10 includes images (A) to (H) illustrating states of cell spheroids before cryopreservation, in which the cell spheroids were produced without supplementing a culture medium with ascorbic acid in the states illustrated in images (A) to (D), the cell spheroids were produced by supplementing a culture medium with ascorbic acid in the states illustrated in images (E) to (H), image (A) is an image of HE staining, image (B) is an image of TUNNEL/DAPI staining, image (C) is a partially enlarged image of image (B), image (D) is an image of immunostaining, image (E) is an image of HE staining, image (F) is an image of TUNNEL/DAPI staining, image (G) is a partially enlarged image of image (F), and image (H) is an image of immunostaining;

FIG. 11 includes images (A) to (F) illustrating states of cell spheroids 5 days after cryopreservation and thawing, in which the cell spheroids were produced without supplementing a culture medium with ascorbic acid in the states illustrated in images (A) to (C), the cell spheroids were produced by supplementing a culture medium with ascorbic acid in the states illustrated in images (D) to (F), image (A) is an image of HE staining, image (B) is an image of TUNNEL/DAPI staining, image (C) is a partially enlarged image of image (B), image (D) is an image of HE staining, image (E) is an image of TUNNEL/DAPI staining, and image (F) is a partially enlarged image of image (E);

FIG. 12 is graph illustrating percentages of cell death in cell spheroids 5 days after thawing; FIG. 13 is a graph illustrating amounts of calcium deposition in C-MSCs cultured in an osteogenic induction medium;

FIG. 14 includes CT images (A) to (D) 4 weeks after transplantation of C-MSCs in a rat calvarium, in which image (A) is an image in which no transplantation has been done, image (B) is an image in which M-MSCs cryopreserved in a DMSO cryopreservation solution have been transplanted, image (C) is an image in which C-MSCs cryopreserved in CELL BANKER (trade name, Takara Bio Inc.) have been transplanted, and image (D) is an image in which C-MSCs cryopreserved in BAMBANKER (trade name, Wako Pure Chemical Industries, Ltd.) have been transplanted;

FIG. 15 includes images (A) to (D) illustrating states of C-MSCs cryopreserved for a long period and thawed, in which image (A) is an image of HE staining, image (B) is an image of TUNEL/DAPI staining, image (C) is a partially enlarged image of TUNEL staining, and image (D) is a partially enlarged image of TUNEL/DAPI staining;

FIG. 16 includes CT images (A) and (B) of a rat calvarium illustrating transplantation effects of C-MSCs, in which image (A) is an image in which no transplantation has been done, and image (B) is an image 4 weeks after transplantation of the C-MSCs; and

FIG. 17 includes images (A) and (B) of HE-stained cross sections of a rat calvarium illustrating transplantation effects of C-MSCs, in which image (A) is an image in which no transplantation has been done, and image (B) is an image 4 weeks after transplantation of the C-MSCs.

### DESCRIPTION OF EMBODIMENTS

A frozen cell cluster according to the present disclosure is a cell cluster in a frozen state in which a plurality of mesenchymal stem cells is granularly formed in cluster form.

The frozen cell cluster can be any cell cluster that has a size in accordance with a form to be used. For example, when the frozen cell cluster is used as a graft for an osteoclastic disease such as bone fracture or periodontitis, the cell cluster preferably has a size that facilitates transplantation into defective tissue in transplant surgery, the size being from 0.5 mm to 1.5 mm in diameter, and more preferably from 0.8 mm to 1.2 mm in diameter.

Note that the term “graft” as used herein refers to all forms that are used for tissue regeneration regardless of techniques, such as, in addition to a form that is directly implanted and used in a bone defective site or the like, a form that is infused into the body with an injection or the like to achieve tissue regeneration.

The frozen cell cluster is thawed before transplant surgery or the like and used in transplant surgery or the like. The frozen cell cluster holds the form thereof even after being thawed, without losing cell functions. Thus, for example, transplantation into defective tissue allows the frozen cell cluster to exert tissue regeneration ability. Note that the frozen cell cluster can be thawed by various techniques, such as allowing a cryopreservation container containing the frozen cell cluster taken out from a freezer or the like to be placed at room temperature or placed in a warm bath (for example, 40° C. to 60° C.).

The frozen cell cluster described above can be produced as follows. First, using a culture vessel such as a culture dish, a collected mesenchymal stem cell is cultured in a growth medium containing a factor that causes the MSC to produce collagen. The mesenchymal stem cell to be used is collected from any of various tissues such as marrow, fat tissue, placental tissue or umbilical cord tissue, and dental pulp.

The growth medium can be any growth medium that allows a mesenchymal stem cell to grow, and a typical commercially available growth medium (for example, Dulbecco's Modified Eagle's Medium (DMEM)+10% fetal bovine serum (FBS)) may be used. Additionally, the factor that causes the MSC to produce collagen is not limited as long as the factor can eventually cause the MSC to produce collagen, such as compounds and proteins or the like. Examples of the factor include, in addition to ascorbic acid, steroids such as dexamethasone, and cytokines, and ascorbic acid is preferred.

When a mesenchymal stem cell grows, a sheet-shaped cell population (hereinafter referred to as cell sheet) is formed. Then, the cells are allowed to grow until the periphery of the cell sheet adheres to the edge of the culture vessel, and the cells reach confluence.

Next, the edge of the cell sheet adhering to the peripheral wall of the culture vessel is separated from the culture vessel. For example, a thin rod is inserted at the inner wall of the culture vessel to which the edge of the cell sheet adheres, and then the thin rod is moved once along the length of the inner wall of the culture vessel so that the cell sheet can be separated from the culture vessel. As a result, the cell sheet floats.

The floating cell sheet rolls up by self-aggregating effect. Then an aggregated mesenchymal stem cell cluster is formed by using an extracellular matrix (ECM) self-produced by the mesenchymal stem cells. In this manner, the granular cell cluster can be obtained.

Note that when a culture vessel that has a cylindrical shape with an area of 1.5 cm² to 2.5 cm² is used, a cell cluster that has a particle diameter of 0.5 mm to 1.5 mm can be obtained. A 24-well plate having a surface area of 2 cm² per well or the like, for example, may be used as the culture vessel.

In addition, in order to obtain a cell cluster in spheroid form, the cell cluster can be produced as follows. An MSC is cultured so as to float in a growth medium, whereby MSCs aggregate with each other and become granular due to intercellular adhesion. After that, a substance that causes MSCs to produce collagen is added to the growth medium. As a result, since the MSCs produce collagen, a cell cluster in spheroid form that is rich in collagen in comparison to normal cell spheroids can be obtained.

The obtained MSC graft is placed together with a cryopreservation agent in a cryopreservation container such as a freezing vial, and is then cryopreserved. A frozen graft can be obtained in this manner.

Various cell cryopreservation solutions may be used as the cryopreservation agent. Examples of the cryopreservation solutions include, in addition to a cryopreservation solution containing 10% dimethyl sulfoxide (DMSO), 20% FBS, and 70% DMEM, and also commercially available cryopreservation solutions such as CELL BANKER (trade name, Takara Bio Inc.) and BAMBANKER (trade name, Wako Pure Chemical Industries, Ltd).

Cryopreservation may be performed at a temperature of −70° C. to −90° C., and preferably −75° C. to −85° C. Additionally, cryopreservation can be performed by any of various techniques such as placing the preservation container in a freezer. In addition, when performing long-term preserving, such as for one month or longer, the preservation container may be placed at the above temperature for 24 hours, and then may be transferred to and preserved in a liquid nitrogen tank (−196° C.).

Pre-freezing may be omitted when performing cryopreservation. Even when cryopreservation is directly performed without pre-freezing, the cell tissue of an MSC graft hardly collapses.

Freezing MSC grafts using an artificial scaffold material (for example, such as hydroxyapatite, tricalcium phosphate apatite, polylactic acid, chitosan, atelocollagen gel, or hyaluronic acid gel) changes physical properties of the artificial scaffold material, thereby hindering the MSC grafts from achieving functions thereof after thawing, and thus cryopreservation is not performed on such MSC grafts using the artificial scaffold material. In addition, cell sheets and cell spheroids, which are used as grafts using no scaffold material, cannot maintain the respective forms thereof after freezing and thawing, as is described in Examples given below, thus heretofore precluding cryopreservation.

However, the frozen cell cluster according to the present disclosure is one prepared by producing, and then cryopreserving, an MSC cell cluster. Thus, MSC cell clusters can be produced from an MSC isolated from a patient, and cell functions and cell uniformity of the MSC cell clusters can be examined in advance. Then, only those MSC cell clusters having constant quality are selected by examination and cryopreserved as frozen cell clusters. Thus, an MSC graft that is the quality-controlled MSC cell cluster can be reliably supplied on the day of transplantation, thereby enabling the prevention of postponement of transplant surgery.

For example, bone marrow mesenchymal stem cells isolated from a patient with periodontitis are transformed into an MSC graft, which is then subjected to examination for cell functions, foreign matter contamination, and the like, and is cryopreserved. In the meantime, periodontal basic treatment, such as removal of infection source and inflammation reduction, which are required before transplantation, are completed, and then, the quality-controlled MSC graft is thawed and transplanted into defective tissue on the day of transplantation, and thus periodontal tissue regeneration can be achieved.

Furthermore, due to low antigenicity of the MSC, the MSC is thought to be a cell applicable to allogeneic transplantation. Accordingly, when an MSC supply system for allogeneic transplantation, such as an MSC bank, is established, cryopreserving MSCs in the form of an MSC cell cluster instead of directly freezing enables implementation of cellular medicine treatment that can provide a favorable MSC cell cluster immediately and reliably when needed for a patient.

### EXAMPLES

### Production and Thawing of Frozen Graft

Mesenchymal stem cells were collected from the bone marrow of a 3-week old rat femur. The collected mesenchymal stem cells were cultured in a growth medium (hereinafter referred to as GM) made using DMEM (Sigma), to which was added 10% FBS (Biowest), 100 U/mL of penicillin (Sigma), 100 μg/mL of streptomycin (Sigma), and 500 ng/mL of amphotericin B (Invitrogen). After 24 hours, non-adherent cells were removed, and adherent cells were further cultured to obtain third passage cells, which cells were used in the following experiment.

The MSCs were seeded on a 24-well culture plate at a ratio of 7.0×10⁴ cells/well, and cultured for 7 hours in a growth medium (DMEM+10% FBS) supplemented with L-ascorbic acid (50 μg/mL).

Due to the culturing, a cell sheet was formed from an MSC/ECM complex by an extracellular matrix produced by the MSCs themselves. Then, after the periphery of the cell sheet contacted with the peripheral wall of the 24-well culture plate and the cells reached confluence, the periphery of the cell sheet was peeled off from the 24-well culture plate by using a micropipette tip. As a result, the cell sheet floated and rolled up further manual assistance.

Then after culturing for 1 day, an MSC graft (hereinafter referred to as C-MSCs) was obtained as a granular cell cluster with a diameter of 0.9 to 1.2 mm.

One cluster (2×10⁵ cells) of the C-MSCs was immersed in 500 μL of a cryopreservation solution, and a 1.5 mL cryovial was used for freezing at −80° C. by placement in a deep freezer without pre-freezing.

Cryopreservation was performed in each of three types of cryopreservation solutions: a cryopreservation solution (hereinafter referred to as DMSO cryopreservation solution) prepared by mixing 10% DMSO, 20% FBS, and 70% DMEM; CELL BANKER (trade name, Takara Bio Inc.); and BAMBANKER (trade name, Wako Pure Chemical Industries, Ltd.).

After freezing for 48 hours, the C-MSCs were quickly defrosted and thawed in a thermostatic water bath set at 37° C. and then were removed from the thermostatic water bath. Then the C-MSCs were immersed in a 24-well culture plate containing 500 μL of a normal medium (DMEM+10% FBS) and were cultured again.

### Verification of Cell Survival Activity

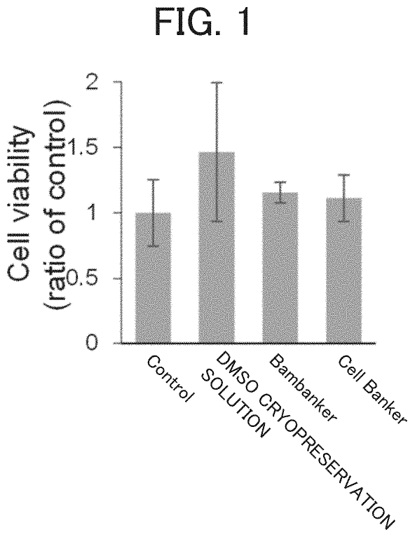

After thawing the respective C-MSCs, cell survival activity was measured using a cell viability kit. FIG. 1 shows the results of such measurement. Non-frozen C-MSCs were measured as a control, and the cell viability of FIG. 1 indicates relative values with respect to the control.

As seen in FIG. 1, the C-MSCs frozen in any of the cryopreservation solutions exhibited no significant difference as compared with the control, and cell survival activity of the frozen C-MSCs was not inferior to those not frozen.

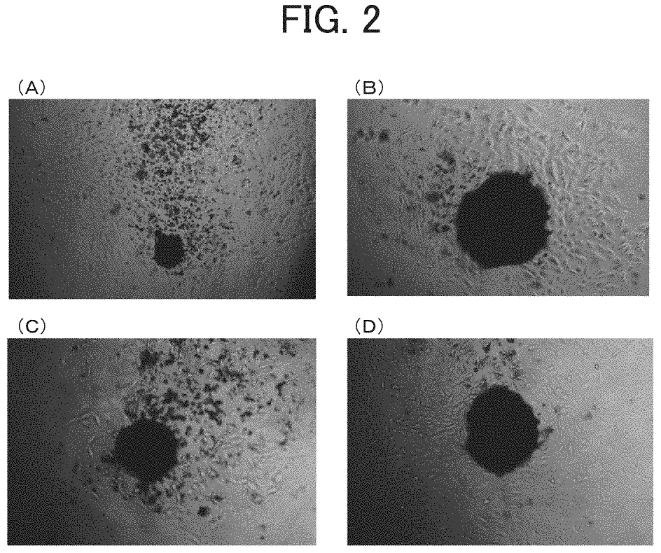

In addition, phase-contract microscopy imaging was used to image each of the C-MSCs. FIG. 2 shows the resultant images. Viewing FIG. 2 confirms that cells were released from all of the C-MSCs. Such release confirmed that normal graft function could be achieved.

### Verification by HE Staining

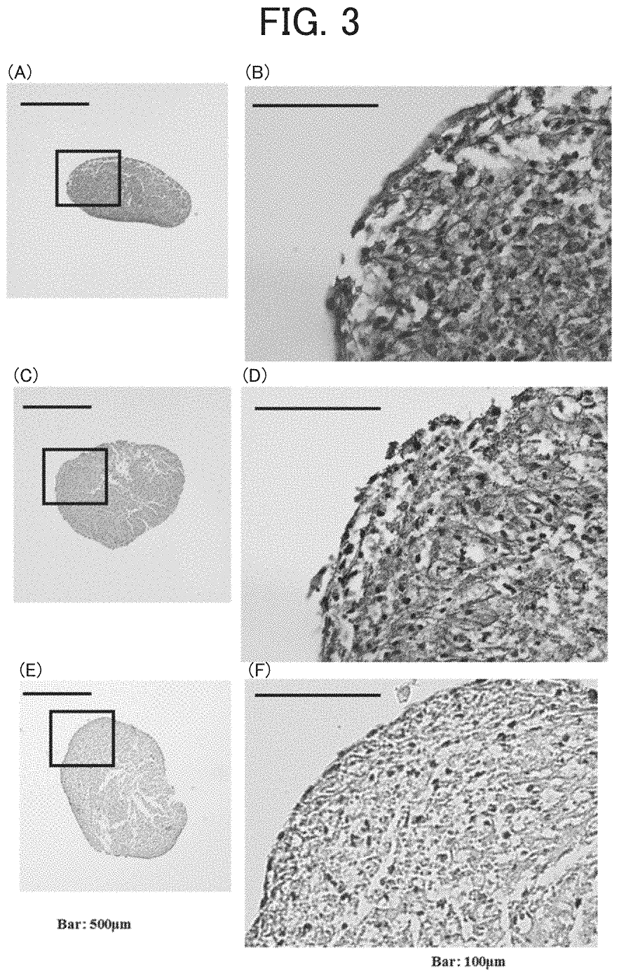

C-MSCs before cryopreservation, 1 day after cryopreservation and then thawing, and 5 days after thawing were hematoxylin-eosin (HE) stained, and HE-stained type imaging was performed to observe tissue morphology. FIG. 3 shows the HE-stained images.

As can be seen in FIG. 3, before cryopreservation, 1 day after thawing, and 5 days after thawing, the morphology of the C-MSCs did not substantially collapse, and the cells and the matrix remained even when the C-MSCs were cryopreserved.

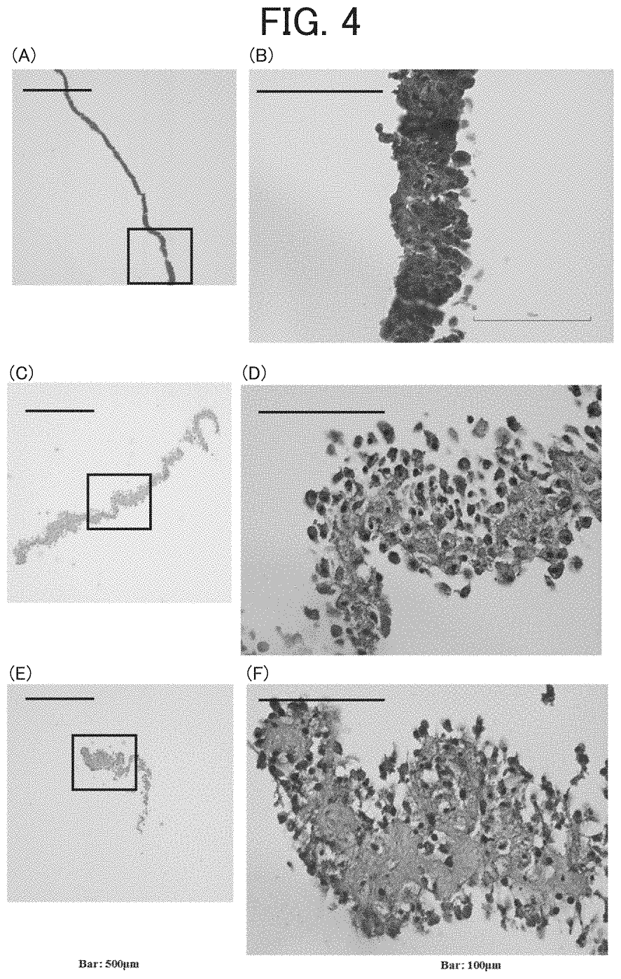

Additionally, a cell sheet (2×10⁵ cells) and a cell spheroid (2×10⁵ cells) were used in place of the C-MSCs to perform cryopreservation using a DMSO cryopreservation solution and then were thawed, similarly to the above. Then HE-stained imaging of the cell sheet and the cell spheroid was performed, similarly to the above. FIG. 4 shows the HE-stained images of the cell sheet, and FIG. 5 shows the HE-stained images of the cell spheroid. Note that the cell sheet and the spheroid used were produced with reference to “Akabane, M. et al., 2008, Journal of Tissue Engineering and Regenerative Medicine” and “Priya, R. et al., 2012, Cell and Tissue Research”, respectively.

FIG. 4 indicates that the morphology of the cell sheet collapsed in a time-dependent manner. Additionally, FIG. 5 indicates that, in the same manner as the cell sheet, the morphology of the cell spheroid collapsed in a time-dependent manner.

Thus due to the collapse of morphology use of the cryopreserved cell sheet and cell spheroid as a graft is understood to not to be possible. In comparison with the C-MSCs, the cell sheet had a coarse matrix, and the matrix (extracellular matrix) of the cell spheroid was small and formed by intercellular adhesion. Thus upon cryopreservation, ice crystals due to freezing seem to have damaged the morphology in both cases.

### Verification by TdT-Mediated dUTP Nick End Labeling (TUNEL) Method

In addition, TUNEL assay was performed on pre-cryopreservation C-MSCs, and also on, after 5 days after cryopreservation and then thawing, C-MSCs, a cell sheet, and a cell spheroid.

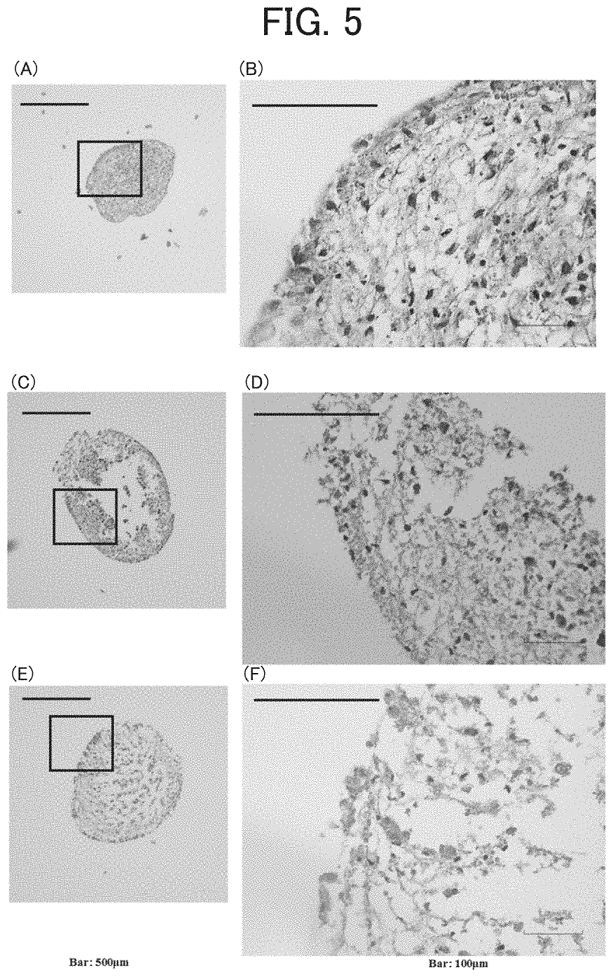

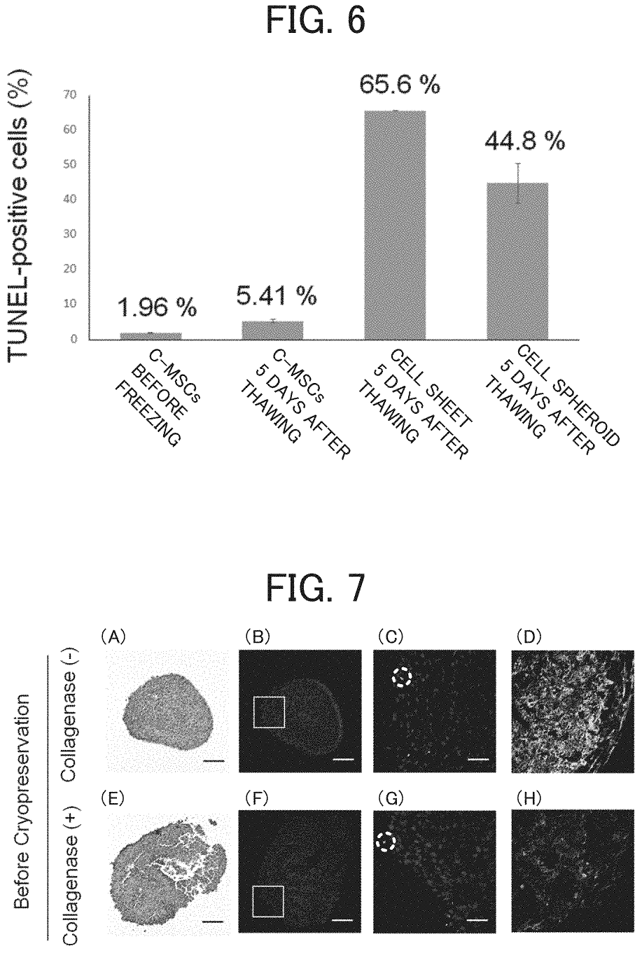

Results are shown in FIG. 6. The percentages of cell death in the C-MSCs before cryopreservation and in the C-MSCs after cryopreservation and thawing were 1.96% and 5.41%, respectively. Even after cryopreservation and thawing, the cell death in the C-MSCs did not increase much. However, in the cell sheet and the cell spheroid after cryopreservation and thawing, the percentages of cell death were very high, 65.6% and 44.8%, displaying much higher percentages than in the C-MSCs.

Thus, cell death was confirmed to hardly occur in C-MSCs even after freezing and thawing, thereby enabling cryopreservation; and many cells were confirmed to have died when the cell sheet and the cell spheroid were frozen, so that cryopreservation is impossible.

### Investigation of Role of Extracellular Matrix in Frozen C-MSCs

Cryopreservation is thought to be enabled by the C-MSCs, in contrast to the cell sheet and the cell spheroid, abundantly producing extracellular matrix by the cells. In order to test this presumption, the following testing examination was performed using an enzyme for decomposing collagen that is the main component of the extracellular matrix.

After producing C-MSCs in the same manner as above, 3 mg/ml of a collagen decomposing enzyme (collagenase (Sigma)) was allowed to act on the C-MSCs for 15 minutes (a collagenase (+) group) immediately before freezing the C-MSCs.

Clusters of the C-MSCs were immersed one by one in 500 μl of a DMSO cryopreservation solution and cryopreserved in the same manner as above. After 48 hours, the C-MSCs were thawed in the same manner as above, and then cultured in 500 μl of GM. Five days after the thawing, the C-MSCs were fixed with 1% formaldehyde, and embedded in paraffin to produce serial sections with thicknesses of 5 μm and 20 μm. The 5 μm-thick sections were HE-stained, and the histological structure of the stained sections was observed through an optical microscope. The 20 μm-thick sections were TUNEL-stained using a DeadEnd Fluorometric terminal deoxynucleotidyl transferase System, and the number of dead cells was observed through a confocal laser scanning microscope.

Additionally, a control experiment was similarly performed using a collagenase (−) group that was not treated with collagenase.

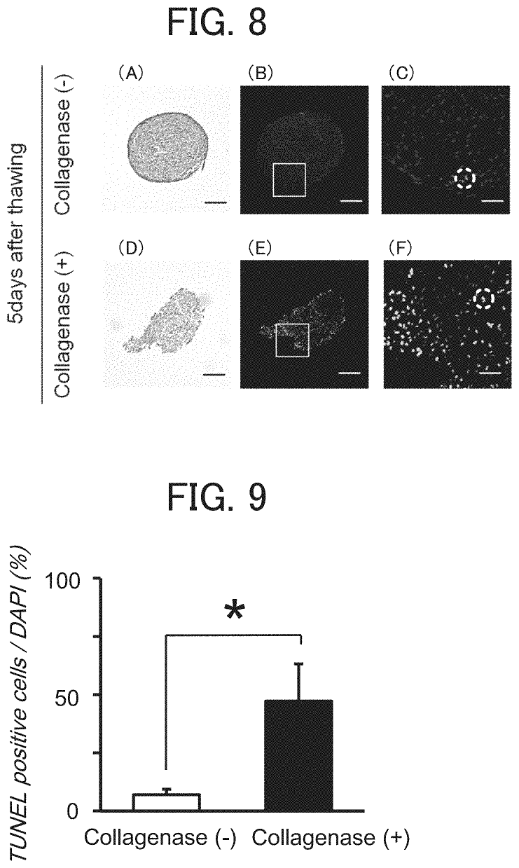

Images (A) to (D) of FIG. 7 and images (A) to (C) of FIG. 8 show states of the collagenase (+) group before cryopreservation and 5 days after thawing, and images (E) to (H) of FIG. 7 and images (D) to (F) of FIG. 8 show states of the collagenase (+) group before cryopreservation and 5 days after thawing. In images (B), (C), (F) and (G) of FIG. 7 and images (B), (C), (E) and (F) of FIG. 8, portions with high brightness (portions enclosed by a broken line, as one example) represent the cell nuclei of dead cells.

As can be seen in image (D) of FIG. 7, the collagenase treatment of the C-MSCs reduced expression level of Type I collagen before cryopreservation. Then, in images (D) to (F) of FIG. 8, morphology of the collagenase (+) group was observed to have collapsed after freezing and thawing, and dead cells were observed to have increased.

However, images (A) to (C) of FIG. 8 indicate that the normal C-MSCs collagenase (−) group retained morphology thereof even after freezing and thawing, and also indicate that the number of dead cells was small.

In addition, FIG. 9 shows the percentages of cell death in the collagenase (−) group and the collagenase (+) group. FIG. 9 indicates a significant difference in the percentages of cell death between the collagenase (−) group and the collagenase (+) group. Such observations suggest that the ECM, which mainly contains collagen, exerts a protective effect against freezing.

### Verification of Cryopreservation of MSC in Cell Spheroid Form Produced by Production of Collagen

C-MSCs can be cryopreserved due to the protective effect of ECM. Thus, supposing that even in the form of a cell spheroid, cryopreservation is possible if collagen is produced from the cells, it was verified whether cryopreservation was possible or not even in the form of a spheroid.

MSCs were seeded on an ultra-low binding 24-well plate at a cell density of 2×10⁵ cells/well, and cultured in GM for 4 days to obtain cell spheroids (a VC (−) group) comprising MSCs.

In addition, after aggregation of the MSCs during the culturing, the GM was supplemented with 50 μg/ml of ascorbic acid (Sigma) to culture and produce cell spheroids (a VC (+) group).

Viability of the cell spheroids before cryopreservation and expression of Type I collagen as ECM were observed by HE staining, TUNEL staining, and immunostaining.

Furthermore, the cell spheroids were immersed one by one in 500 μl aliquots of a DMSO cryopreservation solution, and frozen at −80° C. by using a 1.5 ml cryovial (TrueLine). After 48 hours, the cells were rapidly thawed in a thermostatic water bath set at 37° C., seeded again on a 24-well culture plate, and cultured in 500 μl of GM.

Five days after thawing of the cell spheroids, the cell spheroids were fixed with 1% formaldehyde and embedded in paraffin to produce serial sections with thicknesses of 5 μm and 20 μm. The 5 μm-thick sections were stained with hematoxylin-eosin (HE staining), and the histological structure of the stained sections was observed through an optical microscope. The 20 μm-thick sections were TUNEL-stained using the DeadEnd Fluorometric terminal deoxynucleotidyl transferase System (Promega), and the number of dead cells was observed through a confocal laser scanning microscope.

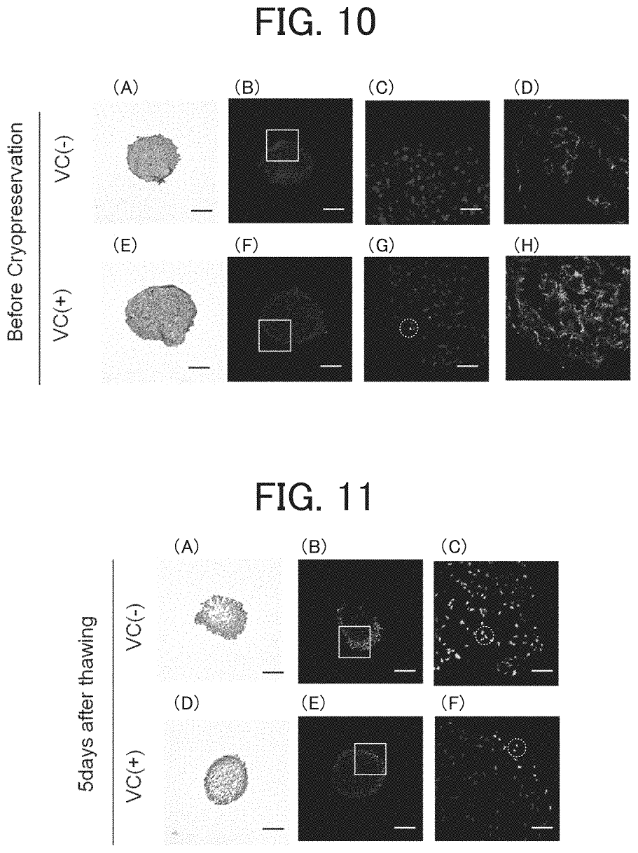

Images (A) to (D) of FIG. 10 and images (A) to (C) of FIG. 11 show states of the VC (−) group before cryopreservation and 5 days after thawing, and images (E) to (H) of FIG. 10 and images (D) to (F) of FIG. 11 show states of the VC (+) group before cryopreservation and 5 days after thawing, respectively. Note that, in images (B), (F) and (G) of FIG. 10 and images (B), (C), (E) and (F) of FIG. 11, portions with high brightness (portions enclosed by a broken line, as one example) represent the cell nuclei of dead cells.

As seen in image (D) of FIG. 10, expression level of Type I collagen was low in the spheroids of the VC (−) group not supplemented with ascorbic acid. Then as seen in images (A) to (C) of FIG. 11, the morphology of the group collapsed after freezing and thawing, and the number of dead cells was observed to increase.

However, as seen in image (H) of FIG. 10, in the spheroids of the VC (+) group supplemented with ascorbic acid, high expression of Type I collagen was observed. Then, as seen in images (D) to (F) of FIG. 11, morphology of the group was retained even after freezing and thawing, and the number of dead cells was small.

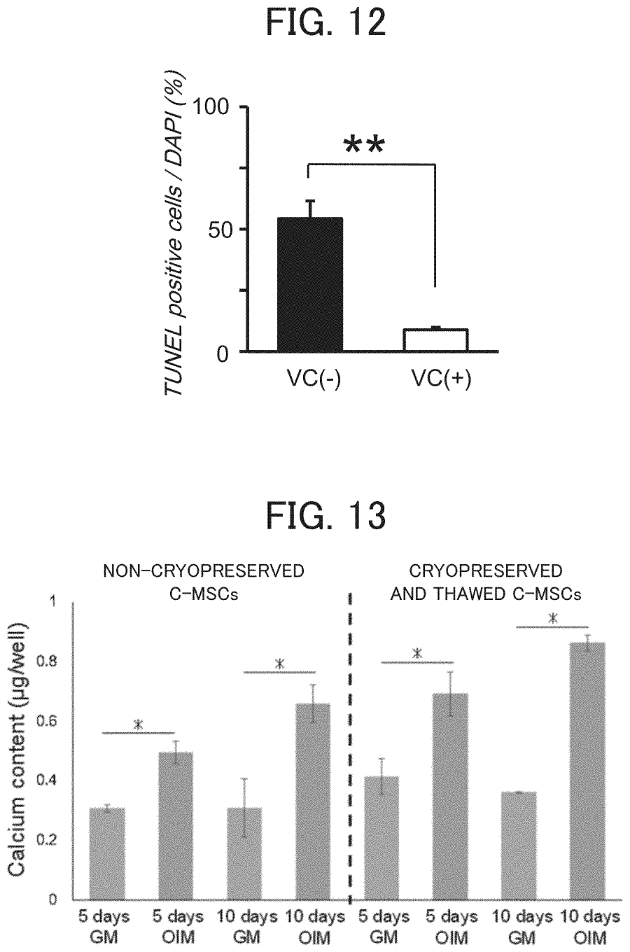

In addition, FIG. 12 indicates the percentages of dead cells 5 days after thawing in the VC (+) group and the VC (−) group. FIG. 12 indicates a significant difference in the percentages of dead cells between the VC (+) group and the VC (−) group. From the above observations, cryopreservation is understood to be possible due to abundant production of the ECM, even in cell spheroids.

### Verification of Osteogenic Differentiation Potency

Subsequently, osteogenic differentiation potency of C-MSCs was verified. Using low-attachment culture dishes, C-MSCs were cultured in normal culture medium (GM) and osteogenic induction medium (OIM), respectively. Then after 5 days and 10 days of culturing, the amount of calcium deposition in the C-MSCs was quantified.

FIG. 13 shows results of quantification. As seen in FIG. 13, in the same manner as the C-MSCs that had not been frozen and thawed, frozen and thawed C-MSCs had increased amounts of calcium deposition due to culturing in the osteogenic induction medium, thus confirming that osteogenic differentiation potency has not been lost.

### Verification of Bone Regeneration

Then verification of bone regeneration by transplantation of C-MSCs was performed. Cryopreserved and thawed C-MSCs were transplanted in a rat calvarial 1.6 mm-radial bone defect model.

Then four weeks after the transplantation, computed tomography (CT) imaging was performed to verify the presence or absence of bone regeneration.

FIG. 14 shows the CT images. In comparison to calvarial bones without transplantation, bone regeneration was induced by the C-MSCs cryopreserved in any of the cryopreservation agents.

### Verification of Influence due to Long-Term Cryopreservation of C-MSCs

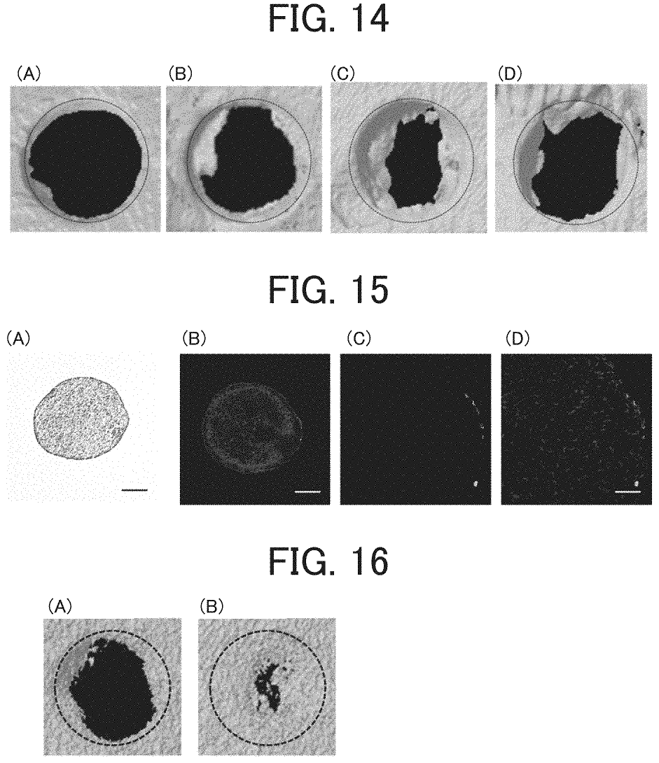

C-MSCs were cryopreserved for 6 months by the same method as above using a DMSO cryopreservation solution. Then, the C-MSCs were thawed by the same method as above. HE-staining, TUNEL-staining, and DIPI-staining of the C-MSCs were performed. Results are illustrated in FIG. 15.

FIG. 15 indicates that even the C-MSCs cryopreserved for 6 months retained morphology, and no significant increase of dead cells was observed.

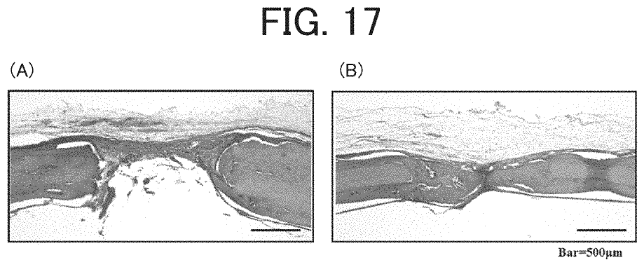

Furthermore, the C-MSCs thawed after the 6-month cryopreservation were transplanted in a rat calvarial defect model by the same method as above, and the presence or absence of bone regeneration was verified. FIG. 16 shows CT images. Additionally, FIG. 17 shows HE-stained cross-sectional images. In comparison to the controls of image (A) of FIG. 16 and image (A) of FIG. 17, image (B) of FIG. 16 and image (B) of FIG. 17 indicate that bone regeneration was promoted in the calvaria with the transplanted C-MSCs. Thus thawing and transplanting the C-MSCs were confirmed to promote bone regeneration even in C-MSCs cryopreserved for a long time of 6 months.

From the above verifications, cryopreserved C-MSCs are proven to not lose functions as a graft even when thawed, and thus can be usable for regenerative medicine by transplant surgery.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

This application claims the benefit of Japanese Patent Application No. 2016-89188, filed on Apr. 27, 2016, the entire disclosure of which is incorporated by reference herein.

### INDUSTRIAL APPLICABILITY

The frozen cell cluster according to the present disclosure is usable in transplantation and regenerative medicine and the like.

What is claimed is:

1. A frozen cell cluster that is a cell cluster in a frozen state in which mesenchymal stem cells are granularly formed.

2. The frozen cell cluster according to claim 1, wherein the frozen cell cluster has a granular shape with a diameter of 0.5 mm to 1.5 mm.

3. The frozen cell cluster according to claim 2, wherein the frozen cell cluster has a granular shape with a diameter of 0.8 mm to 1.2 mm.

4. A method for producing a frozen cell cluster, comprising:
culturing a mesenchymal stem cell in a growth medium containing a factor that causes the mesenchymal stem cell to produce collagen;
forming a granular cell cluster containing the collagen produced from the mesenchymal stem cell; and
isolating the cell cluster from the growth medium and cryopreserving the cell cluster together with a cryopreservation agent.

5. The method for producing a frozen cell cluster according to claim 4, wherein:
a mesenchymal stem cell is cultured to form a cell sheet; and
a periphery of the cell sheet is configured as a free edge to cause forming of a granular cell cluster by a self-aggregating effect.

6. The method for producing a frozen cell cluster according to claim 5, wherein the periphery of the cell sheet is made the free edge by culturing the mesenchymal stem cell in a culture vessel and allowing growth of the mesenchymal stem cell to reach confluence, and separating an edge of the cell sheet adhering to a peripheral wall of the culture vessel from the peripheral wall of the culture vessel.

7. The method for producing a frozen cell cluster according to claim 6, wherein the cell culture is obtained using the culture vessel that has a cylindrical shape having an area of 1.5 cm2 to 2.5 cm2, and the obtained cell cluster has a granular shape with a diameter of 0.5 mm to 1.5 mm.

8. A method for producing a frozen cell cluster, comprising:
culturing a mesenchymal stem cell in a growth medium to cause aggregation;
supplementing the growth medium with a factor that causes the mesenchymal stem cell to produce collagen to form a granular cell cluster containing the collagen produced from the mesenchymal stem cell; and
isolating the cell cluster from the growth medium and cryopreserving the cell cluster together with a cryopreservation agent.

9. (canceled)
